(19) United States
(12) Patent Application Publication
(10) Pub. No.: US 2026/0165774 A1
(43) Pub. Date: Jun. 18, 2026

(54) HAND-HELD ELECTROSURGICAL INSTRUMENT, INSULATING INSERT AND ELECTRODE SUPPORT FOR HAND-HELD ELECTROSURGICAL INSTRUMENT

(71) Applicant: OLYMPUS WINTER & IBE GMBH, Hamburg (DE)

(72) Inventors: Christoph KNOPF, Stockelsdorf (DE); Christian BROCKMANN, Hollenstedt (DE); Jiri LAVICKA, Prerov (CZ); Petr KOMINEK, Prerov (CZ)

(73) Assignee: OLYMPUS WINTER & IBE GMBH, Hamburg (DE)

(21) Appl. No.: 19/532,037

(22) Filed: Feb. 6, 2026

Related U.S. Application Data
(62) Division of application No. 18/234,062, filed on Aug. 15, 2023.
(60) Provisional application No. 63/423,992, filed on Nov. 9, 2022.

(51) Int. Cl. A61B 18/14; A61B 18/00

(52) U.S. Cl. CPC A61B 18/1485; A61B 18/149; A61B 2018/00083; A61B 2018/00601; A61B 2018/00625

(57) ABSTRACT

An insulation insert, an electrode carrier and an electrosurgical handheld instrument which can be used particularly efficiently and which is producible particularly cost-effectively. This is achieved by virtue of the insulation insert having a tube-like embodiment and being detachably couplable by way of a proximal end region to a distal end of a tube-like shaft of the handheld device and a central passage serving to accommodate an inner shaft of the handheld device. Two drilled holes for accommodating a respective electrode carrier tube of an electrode carrier are arranged opposite one another in a wall of the insulation insert and parallel to the central passage.

### CROSS-REFERENCE TO RELATED APPLICATIONS

The application is a divisional application of U.S. application Ser. No. 18/234,062 filed Aug. 15, 2023 the contents of the aforementioned application being incorporated herein by reference.

### DESCRIPTION

The invention relates to an insulation insert for an electrosurgical handheld instrument in accordance with the preamble of Claim 1 and to an electrode carrier for an electrosurgical handheld instrument in accordance with the preamble of Claim 5. Moreover, the invention relates to a method for producing an electrode carrier according to Claim 18 and to an electrosurgical handheld instrument according to Claim 20.

Generic electrosurgical handheld devices, in particular resectoscopes, are used predominantly in urology for electrosurgical work. In this context, these devices are usually used for resection and evaporation of tissue, for example tissue in the lower urinary tract. To this end, the handheld device, in particular the resectoscope, may comprise a longitudinally displaceable electrode carrier, the distal work end of which, following the insertion of the device into the body to be treated, can be advanced from a distal end of the instrument shaft of the handheld device. An electrosurgical electrode is arranged on the electrode carrier at a distal end. By way of example, this electrode may be in form of a loop and, depending on the structure of the instrument, is pushed or pressed through the tissue for the purpose of manipulating the said tissue.

Radiofrequency electric current is applied to the electrode for the aforementioned application. In this case, the electrode should be prevented from making electrical contact with the shaft tube of the handheld device. In the case of such an electrical contact, a short circuit could cause a device defect or could lead to an unpredictable traumatization in the body to be treated. To avoid such short circuits, the handheld devices comprise an electrically insulating insulation insert, also referred to as insulation tip, at their distal end region. In this case, the insulation insert may be fastened either to an inner shaft or shaft tube, in which an electrode carrier is guided, or to the outer shaft of the instrument. Since such handheld devices may also be designed for multiple use and should accordingly be sterilized or autoclaved regularly, the insulation insert is designed to be detachable for cleaning purposes.

The size of the instrument or its cross section is sought to be as small as possible in the case of the handheld instrument for minimally invasive treatment of patients described herein, so that there is as little traumatization of the patient during the treatment as possible. Similarly, undertaking the intervention is sought to be particularly efficient. The choice of electrode is of decisive importance for an efficient intervention. The optimal treatment goal can only be achieved with the correct, application-specific electrode. Especially the effective cross section of the electrode or work instrument relative to the cross section of the instrument may be of decisive importance. However, the effective cross section or size of the electrode is restricted by the shape and diameter of the shaft of the electrosurgical handheld instrument. Thus, it is not feasible for the effective cross section of the electrode to be greater than the cross section of the outer circumference of the shaft. However, the space available for the electrode is not exploited optimally in the known instruments. Approaches for a better exploitation of the available space pursue highly complicated electrode geometries, which firstly are very complex and hence expensive in terms of production and secondly require much outlay in quality control.

The invention is therefore based on the problem of creating an insulation insert, an electrode carrier and an electrosurgical handheld instrument which can be used particularly efficiently and which is producible particularly cost-effectively.

A solution to this problem is described by Claim 1. Accordingly, provision is made for the insulation insert according to the invention to have a tube-like embodiment and be detachably couplable by way of a proximal end region to a distal end region of a tube-like shaft of the handheld device and for a central passage to serve to accommodate an inner shaft of the handheld device. This inner shaft can be a shaft for accommodating a tool or an optical unit. According to the invention, provision is made for two drilled holes for accommodating a respective electrode carrier tube of an electrode carrier to be arranged opposite one another in a wall of the insulation insert and parallel to the central passage. The stability of the electrode carrier and hence of the electrode as well is improved by accommodating the electrode carrier tubes by way of the two drilled holes in the insulation insert. The stability of the at least one electrode carrier transversely to a longitudinal axis of the handheld instrument is improved by mounting the distal end regions of the electrode carrier in the drilled holes. The electrode can be inserted particularly precisely and hence efficiently as a result of this guidance of the electrode carrier in the drilled hole.

In particular, provision is made for the two drilled holes to have an oval cross section, with a height of the oval cross section being greater than a width of the oval cross section. Moreover, provision is preferably made for the two drilled holes to be displaced preferably upwardly vis-à-vis a central longitudinal axis or central drilled hole. The positions of the electrode carrier tubes which are guided through the insulation insert and through the handheld instrument are also displaced by the oval form of the drilled holes and the upward or downward displacement from the central position of the drilled holes. As a result of this displacement and the oval form of the drilled holes, it is possible to use electrodes which, while having the same width, have a greater height than previous electrodes. As a result of this configuration of the drilled holes, it is possible to use electrodes with a greater effective cross section relative to the cross section of the insulation insert.

A further advantageous exemplary embodiment of the invention may provide for the wall of the insulation insert to be strengthened around the two drilled holes and otherwise be reduced in terms of its wall strength. In the region of the drilled holes, the insulation insert formed otherwise with a thin wall has two inwardly pointing bulges, by means of which the inner, clear cross section of the insulation insert is minimally reduced. As a result of the upward displacement of these bulges out of the central edge region in particular, it is possible to make space in the central interior of the insulation insert.

According to the invention, provision can be made for the insulation insert formed from an electrically insulating material, for example a temperature-stable and plasma-stable plastic, to have coupling means in order to be detachably connected to the shaft. When assembling the handheld instrument, the electrode carrier with the two electrode carrier tubes is guided through the drilled holes and is then detachably fastened to the distal end of the shaft. The proximal ends of the electrode carrier tubes can be connected to a main body of the handheld instrument. To take the handheld instrument apart, the aforementioned steps are carried out in the reverse sequence.

An electrode carrier for solving the stated problem has the features of Claim 5. Accordingly, the electrode carrier for an electrode of an electrosurgical handheld instrument has at least one, preferably two electrode carrier tubes, with the electrode being arrangeable at a distal end of the at least one electrode carrier tube. This electrode carrier is couplable by way of a proximal end of the at least one electrode carrier tube to a main body of the handheld instrument. The electrode carrier can be moved axially and have radiofrequency electric current applied thereto via this main body. According to the invention, provision is made for a cross section of the at least one electrode carrier tube to be oval or convex, especially over its entire length. This oval embodiment of at least a portion of the electrode carrier tube leads to increased stability vis-à-vis forces acting on the electrode carrier tube transversely to the longitudinal axis of the handheld instrument. Moreover, the oval shape leads to the at least one electrode carrier tube needing less space within an instrument shaft. Moreover, it is possible to optimize the position of the distal ends of the at least one electrode carrier tube, and hence of the electrode.

Provision is preferably made for a distal portion of the at least one electrode carrier tube to have an oval or convex cross section. The remainder of the electrode carrier tube may continue to have a circular or any desired cross section. In this case, provision is made for the at least one electrode carrier tube to be oriented in such a way that a height of the oval cross section is greater than a width of the cross section, with the height being a dimension perpendicular to a horizontal plane.

The height-to-width ratio is preferably 1.1:1 to 1.7:1, preferably 1.4:1, to be precise over the entire length or only over the distal portion. It was found that this dimensioning of the at least one electrode carrier tube can be positioned in particularly space-saving fashion within the instrument shaft. This width-to-height ratio represents an optimal compromise between stability and repositioning of the electrode at the distal end of the electrode carrier. A preferred measure for the height is 1.4 mm, and it is 1 mm for the width. However, according to the invention, provision is also made for the absolute dimensions of the oval cross section to deviate at least slightly from these values. As a result of the oval shape of the cross section, it is possible to upwardly displace the at least one electrode carrier tube from the centre of the instrument, without this changing the spacing of the two distal ends of the electrode carrier tubes. As a result of the oval shape, the distal end regions of the tubes can be moved upwardly with an unchanging spacing relative to the central axis of the handheld instrument, with the result that the loop dimension of the electrode is increased, to be precise without projecting beyond the cross section of the handheld instrument. Consequently, this reshaping of the electrode carrier tubes allows work to be carried out with an electrode which has an optimized work cross section.

A preferred exemplary embodiment of the invention provides for the distal portion of the at least one electrode carrier tube to have a length of 20 mm to 50 mm, preferably of 24 mm to 40 mm, and be longer than 30 mm in particular. Here, it is specifically this distal portion that has an oval cross section. The length of this portion corresponds at least to the travel of the electrode carrier within the instrument shaft.

Preferably, the invention can further provide for a transition between a proximal portion of the at least one electrode carrier tube and the distal portion to be formed by a crimp. This crimping represents a local reshaping of the outer circumference of the electrode carrier tube to form a hexagonal cross section. Accordingly, the diameter of the electrode carrier tube is reduced in certain regions. This reshaping of the tube serves for a defined transition from the tube portion with a circular cross section to the tube portion with an oval cross section. Moreover, the reshaping serves to fix the electrical conductor or the wire within the tube.

The hexagonal crimp of the at least one electrode carrier tube is aligned so that two opposite side faces of the hexagonal cross section are aligned parallel to one another and perpendicular to a horizontal plane. As a result of this orientation of the crimp, the maximum diameter of the crimped tube portion does not protrude beyond the oval diameter of the distal portion, with the result that the electrode carrier cannot catch during the forward and backward motion relative to the longitudinal axis of the instrument.

Moreover, provision is preferably also made for a distal end of the at least one electrode carrier tube to have a preferably hexagonal crimp. This crimp allows the interior of the tube to be sealed water-tightly, and the inner wire can thus be protected against liquids flowing in. In this case, provision can be made for this hexagonal reshaping of the cross section to also be aligned in the same way as the crimp between the portion with the round cross section and the portion with the oval cross section.

A further particularly preferred exemplary embodiment of the invention may provide for the at least one electrode carrier tube to comprise at least one, preferably two reflexed profiles, specifically a proximal reflexed profile and a distal reflexed profile, a portion of the electrode carrier tube being displaced in parallel relative to another portion of the electrode carrier tube as a result of this at least one reflexed profile. This reflexed profile allows the distal end, to which the electrode is fastened, of the at least one electrode carrier tube to be displaced relative to the central axis through the instrument. This displacement of the distal end region is possible, in particular, because the distal end region has the above-described oval cross section. Consequently, it is possible that the reflexed profile only needs to be implemented in one dimension and not in two. Accordingly, the relative spacing between the two distal ends of the electrode carrier tubes guided in parallel does not change as a result of the reflexed profile. Only the two distal ends are displaced upwardly relative to the central axis. On account of the modified cross section of the distal end region, this does not lead to a collision of the electrode carrier tubes with the instrument shaft. As a result of this displacement of the distal ends, it is possible to use electrodes which have a larger effective cross section, without these protruding beyond the cross section of the instrument shaft in the process.

Provision is preferably made for the at least one reflexed profile, in particular the distal reflexed profile, to have a height of 0.2 mm to 2 mm, preferably of 0.7 mm. The length of the at least one reflexed profile can be 2 mm to 20 mm, preferably 5 mm to 10 mm.

Moreover, provision can be made according to the invention for the distal portion of the at least one electrode carrier tube with the oval or convex cross section to have at least one distal reflexed profile. In the case where the at least one electrode carrier tube has two reflexed profiles, specifically a distal reflexed profile and a proximal reflexed profile, provision can be made for these two reflexed profiles to be located in a common plane. Equally, it is also conceivable that the two planes of the reflexed profiles are twisted relative to one another.

A method for solving the stated problem has the measures of Claim 18. Accordingly, the method for producing an electrode carrier for an electrode of an electrosurgical handheld instrument having at least one, preferably two electrode carrier tubes according to Claim 5 consists in that a cross section of a distal portion of the electrode carrier tube is reshaped to be oval (step A) and/or in that a transition from a portion with a circular cross section to a portion with an oval cross section is crimped (step B) and/or in that the distal region with the oval cross section is provided with at least one reflexed profile (step C). According to the invention, provision is made for steps A, B and C to be implemented successively or simultaneously.

An electrosurgical handheld device for solving the stated problem has the features of Claim 20. In this case, the electrosurgical handheld device can be, in particular, a resectoscope or the like. The handheld device comprises a main body to which a tube-like shaft is connected. An insulation insert according to Claims 1 to 4 is arrangeable at a distal end of this shaft, with an electrode carrier according to Claims 5 to 17 extending through the shaft and through the insulation insert, the said electrode carrier being fastened to the main body with a proximal end. An electrode is arrangeable at the distal end of the electrode carrier or electrode carrier tube.

A preferred exemplary embodiment of the invention is described in detail below with reference to the drawing, in which:

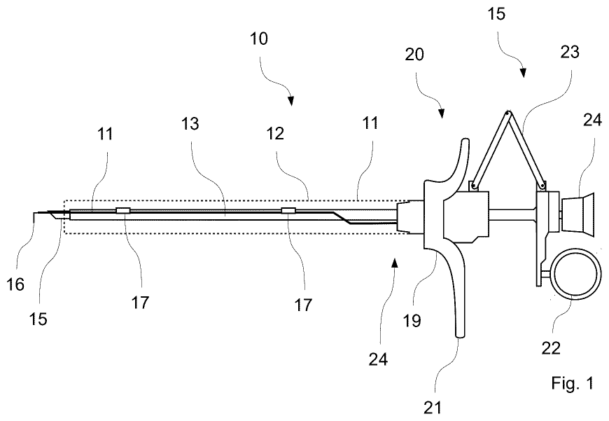

FIG. 1 shows a schematic illustration of a surgical handheld device, in particular a resectoscope,

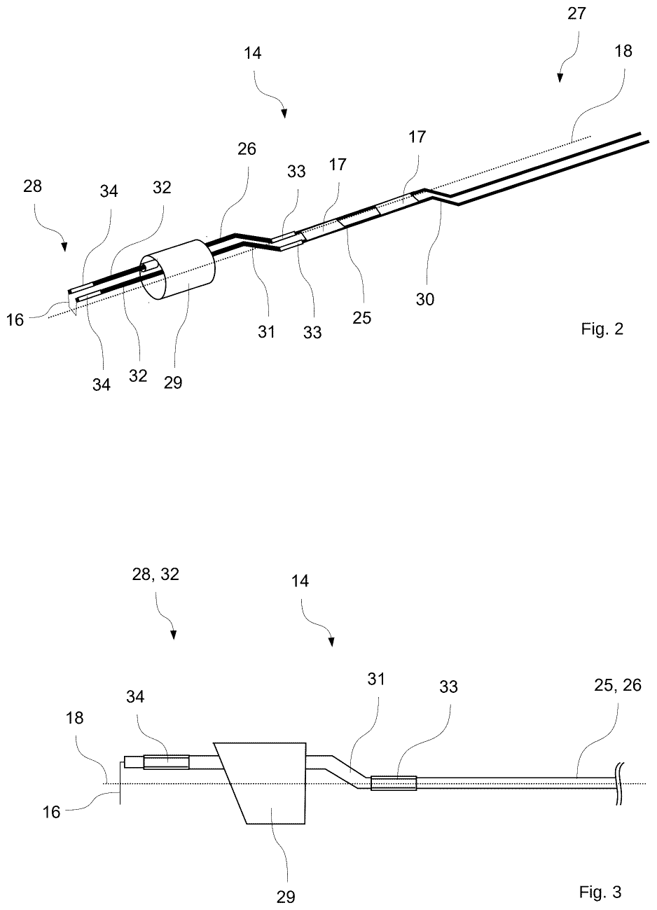

FIG. 2 shows a perspective illustration of an electrode carrier,

FIG. 3 shows a side view of a distal portion of the electrode carrier according to FIG. 2,

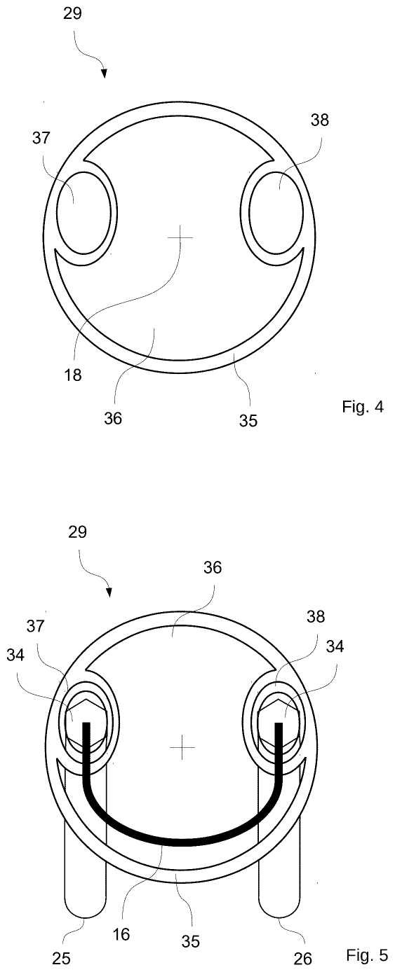

FIG. 4 shows a view of an insulation insert, and

FIG. 5 shows a view of the insulation insert according to FIG. 4 with the electrode carrier.

FIG. 1 shows a schematic, lateral sectional illustration of a known resectoscope 10. The resectoscope 10 has a resectoscope shaft 11, which comprises an illustrated outer shaft 12 or enveloping tube. A tube-like inner shaft 13 extends within the outer shaft 12. An electrode carrier 14 and an indicated optical unit 15 are illustrated within the inner shaft 13. Moreover, further elements (not illustrated here) may be arranged within the resectoscope 10, for example a separate rinsing tube and the like.

The electrode carrier 14 has an electrosurgical tool or electrode 16 at a distal end. The electrode 16 illustrated here is represented as a loop, but it may also be formed as a button or the like.

The electrode carrier 14 can be moved axially in the distal and proximal direction in positively guided fashion by the actuation of a handle 19. In the process, it may be pushed beyond the distal end of the inner shaft 13 and outer shaft 12. This allows the surgeon to also manipulate tissue removed further away from the resectoscope tip. For this purpose, the inner shaft 13 and/or the electrode carrier 14 may further be mounted rotatably about their longitudinal axis. Radiofrequency electric current is applied to the electrode 16 for the manipulation of the tissue.

The resectoscope 10 illustrated in FIG. 1 has a passive transporter, in which a carriage 20 is displaced in the distal direction against the distal, first grip part 21 counter to a spring force applied by a spring bridge 23 as a result of relative movement between the grip parts 21 and 22 arranged proximally at the resectoscope shaft 11. When the carriage 20 is displaced in the distal direction against the grip part 21, the electrode carrier 14 is displaced in the distal direction (in a manner not illustrated here). When pressure is removed from the handle parts 21, 22, the spring force produced by the spring bridge 23 forces the carriage 20 back into its initial position, with the electrode carrier 14 being pulled in the proximal direction. When the carriage 20 is displaced backwards, an electrosurgical intervention can be carried out without a manual force applied by the surgeon, which is to say passively, using the electrode 16.

For the targeted treatment by means of the electrode 16, the optical unit 15 is positioned in such a way that the surgeon has an optimal view of the operation region. To this end, the resectoscope 10 has at a proximal end an eyepiece 24 which is connected to the optical unit 15. Alternatively, it is also conceivable that a camera is arranged at the resectoscope 10 instead of the eyepiece 24.

The electrode carrier 14 consists substantially of two parallel electrode carrier tubes 25, 26 (FIG. 2). These electrode carrier tubes 25, 26 serve to hold the electrode 16 and supply the latter with electrical energy. To this end, an electrical conductor runs from the proximal end 27 to the distal end 28 in at least one of the electrode carrier tubes 25, 26. One of the two electrode carrier tubes 25, 26 is latched with the proximal end 27 in the carriage 20 and connected to a cable which is in contact with an RF generator. The respective other electrode carrier tube 25, 26 is likewise latched in the carriage 20 and forms the neutral electrode. To provide additional stabilization of the elongate electrode carrier tubes 25, 26, these can be interconnected by way of guiding elements 17. These guiding elements 17 also serve to clamp the electrode carrier 14 to the inner shaft 13 or under the inner shaft 13 or the optical unit 15. Moreover, the electrode carrier tubes 25, 26 are guided through an insulation insert 29 and thereby likewise stabilized vis-à-vis transverse forces.

It is essential to the functionality of the resectoscope that the electrode carrier 14, together with the electrode 16, can be retracted completely into the outer shaft 12. To this end, the effective cross section or outer cross section of the electrode 16 may not be greater than the internal diameter of the distal region of the outer shaft 12. Accordingly, the electrode carrier tubes 25, 26 are known to have a reflexed profile 30. This reflexed profile 30 results in two parallel, successive portions along the electrode carrier tubes 25, 26 being displaced in parallel relative to the longitudinal axis 18, and so the distal ends 28 of the electrode carrier tubes 25, 26 are removed from the longitudinal axis 18. To optimize the shape of the electrode 16 and increase the space in the interior of the shaft 13, the invention provides for the electrode carrier tubes 25, 26 to have a second reflexed profile 31. As a result of this second reflexed profile 31, two distal portions 32 of the electrode carrier tubes 25, 26 are removed even further from the longitudinal axis 18 than was already the case due to the reflexed profile 30. Moreover, the invention provides for the distal portions 32 of the electrode carrier tubes 25, 26 to have an oval cross section in contrast with the remaining portions of the electrode carrier tube 25, 26. This oval cross section is designed so that a height of the cross section perpendicular to a horizontal plane is greater than the width of the cross section. This portion with the oval cross section comprises both the distal portion 32 and the reflexed profile 31. The remaining portions of the electrode carrier tubes 25, 26 furthermore have a circular cross section. For a defined transition of the portions with a circular cross section to the portions with an oval cross section, the electrode carrier tubes 25, 26 each have a crimp 33 or an embossment or a deformation. This crimp 33 moreover fixes the electrical conductor within the electrode carrier tubes 25, 26 (FIG. 3).

Moreover, the invention also provides for the distal ends of the electrode carrier tubes 25, 26 to have a crimp 34 or an embossment or a deformation. This hexagonally formed crimp 34 is aligned so that two parallel side faces of the crimp 34 run transversely to a horizontal plane through the electrode carrier tubes 14. As a result, the cross section of the crimp 34 behaves similarly to the oval cross section of the electrode carrier tubes 25, 26. Consequently, the electrode carrier tubes 25, 26 can be retracted completely without the crimp 34 jamming in the insulation insert 29. Express reference should be made here to the fact that the insulation insert 29 is depicted very schematically in FIGS. 2 and 3 and merely serves for elucidation.

FIGS. 4, 5 depict a frontal view of the insulation insert 29 according to the invention. To prevent the electrode 16, to which a voltage has been applied, from coming into contact with the metallically conductive outer shaft 12, the practice of arranging an electrically insulating tip at the distal tip of the inner shaft 13 is known. This usually tube-like tip consists of electrically non-conductive material, for example a plasma-stable and temperature-stable plastic. The insulation insert 29 can be detachably coupled to the shaft 13 by means of coupling elements.

The insulation insert 29 according to the invention, schematically illustrated here, likewise has a tube-like form and a thin wall 35. This wall 35 comprises a central passage 36, through which for example the optical unit 15 and other tools can be guided. Moreover, the insulation insert 29 has two drilled holes 37, 38. These drilled holes 37, 38 are aligned parallel to one another and parallel to the longitudinal axis 18 but are upwardly displaced vis-à-vis the longitudinal axis 18, with the result that the drilled holes 37, 38 are not located centrally or not located in the same horizontal plane as the longitudinal axis 18. The drilled holes 37, 38 serve to accommodate the two electrode carrier tubes 25, 26. To accommodate the electrode carrier tubes 25, 26, the drilled holes 37, 38 likewise have an oval form, with the height of the drilled holes 37, 38 being greater than their width. Only the oval embodiment of the drilled holes 37, 38, the oval cross section of the electrode carrier tubes 25, 26 and the second reflexed profile 31 makes it possible to move the two drilled holes 37, 38 vis-à-vis the longitudinal axis 18, to be precise without the spacing of the two drilled holes 37, 38 having to be modified in the process. This also allows the electrode 16 to maintain its known width, and so it need not be modified. Rather, this reshaping or displacement allows the effective cross section of the electrode 16 to be increased, by virtue of the width being maintained and the length or height of the electrode 16 being adapted to the dimension of the resectoscope 10. Since the spacing of the two drilled holes 37, 38 has not been changed, it is also possible to use current electrodes 16 with the electrode carrier 14.

FIG. 5 depicts the insulation insert 29 with the two electrode carrier tubes 25, 26, which are guided through the two drilled holes 37 and 38. Here, it becomes particularly clear that the crimps 34 of the distal ends of the electrode carrier tubes 25, 26 do not protrude beyond the diameter of the oval cross sections of the distal portions 32 and that the electrode carrier tubes 25, 26 are consequently freely displaceable through the drilled holes 37, 38 of the insulation insert 29.

### LIST OF REFERENCE SIGNS

10 Resectoscope
11 Resectoscope shaft
12 Outer shaft
13 Inner shaft
14 Electrode carrier
15 Optical unit
16 Electrode
17 Guiding element
18 Longitudinal axis
19 Handle
20 Carriage
21 Grip part
22 Grip part
23 Spring bridge
24 Eyepiece
25 Electrode carrier tube
26 Electrode carrier tube
27 Proximal end
28 Distal end
29 Insulation insert
30 Reflexed profile
31 Reflexed profile
32 Distal portion
33 Crimp
34 Crimp
35 Wall

1. Method for producing an electrode carrier for an electrode of an electrosurgical handheld instrument having at least one electrode carrier for an electrode of an electrosurgical handheld instrument wherein:
the at least one electrode carrier comprises at least one electrode carrier tube, with the electrode being arrangeable at a distal end of the at least one electrode carrier tube and the electrode carrier being couplable via a proximal end of the at least one electrode carrier tube to a main body of the handheld instrument; and
a cross section of a distal portion of the electrode carrier tube is reshaped to be oval (step A) and/or a transition from a portion with a circular cross section to a portion with an oval cross section is embossed or crimped (step B) and/or the distal region with the oval cross section is provided with at least one reflexed profile (step C).

2. Method for producing an electrode carrier according to claim 1, wherein steps A, B and C are implemented successively or simultaneously.
